(19) United States
(12) Patent Application Publication
(10) Pub. No.: US 2004/0219582 A1
(43) Pub. Date: Nov. 4, 2004

(54) Single nucleotide polymorphism in the FGF-3 gene and methods of use thereof

(76) Inventor: Yongjun Guo, Wayne, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/798,652

(22) Filed: Mar. 11, 2004

Related U.S. Application Data
(60) Provisional application No. 60/455,689, filed on Mar. 17, 2003.

(51) Int. Cl. C12Q 1/68; C07H 21/04; C12N 9/16

(52) U.S. Cl. 435/6; 435/320.1; 536/23.2; 435/325; 435/196

(57) ABSTRACT

A single nucleotide polymorphism (SNP) in the 5′ untranslated region of the FGF-3 gene, which indicates cancer susceptibility is disclosed. Also disclosed are methods and kits for detecting this SNP in the FGF-3 gene.

This application claims priority under 35 U.S.C. §119 (e) to U.S. Provisional Application No. 60/455,689, filed Mar. 17, 2003, the entire content which is incorporated by reference herein.

### FIELD OF THE INVENTION

The presence invention relates to the discovery of a single nucleotide polymorphism (SNP) in the FGF-3 gene and methods of use thereof.

### BACKGROUND OF THE INVENTION

All publications, patent applications, patents, and other citations mentioned herein are incorporated by reference in their entirety.

An individual's susceptibility to cancer is governed by the individual's genome and carcinogenic stimuli encountered in the environment. Although the carcinogenic potential of many compounds and other stimuli (e.g. ionizing radiation) have been determined, assessing the importance of limiting exposure to such compounds is complicated by the fact that not all individuals are equally susceptible to their deleterious effects. Accordingly, the genetic component of carcinogenic susceptibility confounds accurate prediction of cancer rates among individuals, even in defined environments.

The FGF-3 gene encodes a secreted protein belonging to the fibroblast growth factor (FGF) family (1). FGF-3 was originally identified as a proto-oncogene (at first designated int-2) transcriptionally activated by proviral integration in virally induced mouse mammary tumors (2, 3). In a colon carcinoma cell line, it has been shown that the FGF-3 gene is constitutively expressed in cells from tumorigenic clones but is completely silent in non-tumorigenic clones (4). Amplification of FGF-3 gene has been observed in human esophageal (5-10), breast (11-22), ovarian (17, 18, 23-29), prostate (30-32), and head and neck cancer (5, 33-39). Earlier studies have revealed that 62% of primary esophageal cancers and 100% of these cancers which had metastasized to the lymph node had amplification of FGF-3 gene as demonstrated by Southern blot hybridization.

In 1999, Djenabi et al. (40) published the 5′ untranslated region and promoter sequence upstream of the human FGF-3 gene (GenBank Accession No. Y12377). In 2000, Galdemard et al. identified a putative minimal promoter in the 5′-proximal region of FGF-3, about 6 kb upstream of the FGF-3 gene (41). Additionally, it has been previously demonstrated that this minimal promoter of FGF-3 shares about 45% of sequence homology with the human ODC promoter region.

### SUMMARY OF THE INVENTION

The present invention provides an isolated nucleic acid molecule having the sequence of SEQ ID NO: 1 or a complementary sequence thereof, a vector comprising the same, and a host cell comprising such a vector.

Also provided is a method for detecting a single nucleotide polymorphism (SNP) in the FGF-3 gene in a mammal, which comprises a) isolating a nucleic acid sample from the mammal; and b) determining whether a cytosine or thymine is present at position 69 of SEQ ID NO: 1. In a particular embodiment, the method further comprises amplifying a reference portion of the mammal's genome, which reference portion comprises the nucleotide residue located at position 69 of SEQ ID NO: 1. In a further embodiment, the method also comprises the step of annealing one or two oligonucleotide probes with a target portion of the mammal's genome prior to amplifying the reference portion, which target portion includes the nucleotide residue located at position 69 of SEQ ID NO: 1. Fluorescent label(s) and optionally fluorescent quencher(s) may be attached to the one or both oligonucleotide probes.

Also provided by the present invention is a kit for detecting a SNP in the FGF-3 gene in a mammal, which comprises a) at least one optionally detectably labeled oligonucleotide probe that anneals specifically with a target portion of the mammal's genome; and b) a pair of primers for amplifying a reference portion of the FGF-3 gene, which reference portion includes nucleotide residue located at position 69 of SEQ ID NO: 1.

The present invention further provides a method for assessing the relative susceptibility of a mammal to cancer, which comprises the detection of a SNP in the FGF-3 gene in a mammal. The presence of a cytosine at position 69 of SEQ ID NO: 1, being indicative of a greater susceptibility to the cancer than a mammal which does not comprise a cytosine at the position. Such cancers include, but are not limited to, esophageal, breast, ovarian, prostate, and head and neck cancer.

### BRIEF DESCRIPTION OF THE DRAWINGS

FIG. 1 is a nucleotide sequence showing the variant nucleotide at position 69 in SEQ ID NO: 1 (i.e., the C or T allelle) present in the upstream untranslated region of the FGF-3 gene, extending from nucleotide residues 4945 to 5508, relative to the published sequence of FGF-3 gene upstream flanking region (GenBank Accession No. Y12377). The SNP is located at position −6693 relative to the ATG start codon for FGF-3.

FIG. 2 is a diagram depicting an embodiment of the allelic discrimination method of the invention. “R” (reporter) refers to a fluorescent label (e.g. a dye such as VIC, FAM, TET, JOE, or HEX), and “Q” (quencher) refers to a fluorescence quencher (e.g. TAMRA).

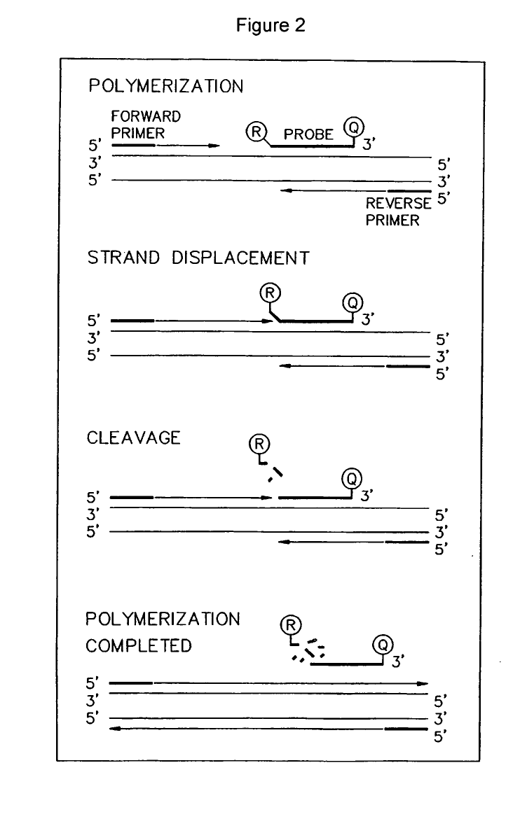

FIG. 3 is a graph depicting the distribution of human FGF-3 genotypes in different ethnic groups, i.e., African American, Caucasian, and Chinese.

### DETAILED DESCRIPTION OF THE INVENTION

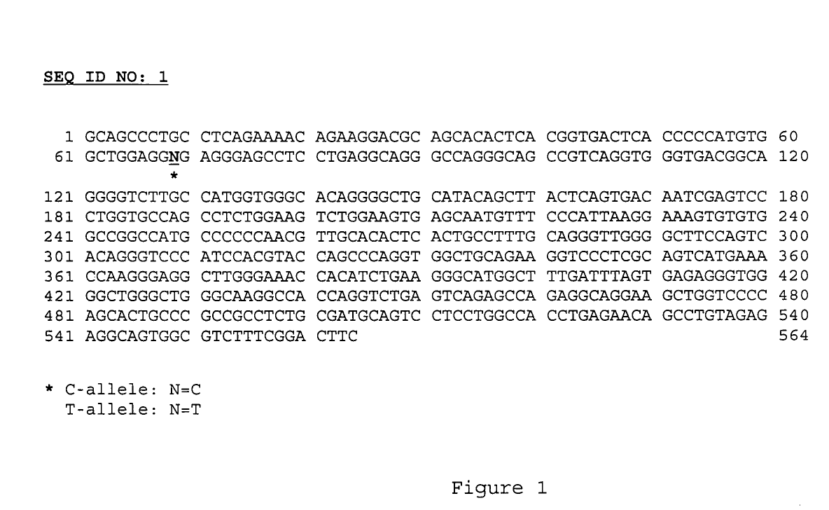

The present invention is based on the discovery of a single nucleotide polymorphism (SNP) in the 5′-proximal region of the FGF-3 promoter, in which a cytosine is substituted for a thymine at position 69 in SEQ ID NO: 1 (FIG. 1). The SNP occurs at position −6693 relative to the ATG codon of the FGF-3 gene. The polymorphism is believed to create a new transcription factor binding sequence in the FGF-3 promoter region. The presence of the C-allele in the FGF-3 gene in an individual is predictive of a predisposition to certain types of cancers, including without limitation, environmentally-induced esophageal squamous cell carcinoma.

### I. Definitions

Various terms relating to the biological molecules of the present invention are used hereinabove and also throughout the specifications and claims.

As used herein, a mammal “comprises a C-allele or T-allele of the FGF-3 gene” if the genome of the mammal comprises one or more copies of the C-allele or T-allele.

A mammal is “homozygous” for the C-allele or T-allele of the FGF-3 gene if the genome of the mammal comprises two copies of the C-allele or T-allele.

The “C-allele” of the FGF-3 gene refers to a mammalian FGF-3 gene having a cytosine residue at position 69 in SEQ ID NO: 1, or at the position −6693 bp upstream of the ATG codon of FGF-3 gene.

The “T-allele” of the FGF-3 gene refers to a mammalian FGF-3 gene having a thymine residue at position 69 in SEQ ID NO: 1, or at the position −6693 bp upstream of the ATG codon of FGF-3 gene.

With reference to nucleic acids of the invention, the term “isolated nucleic acid” or “isolated polynucleotide” is sometimes used. This term, when applied to DNA, refers to a DNA molecule that is separated from sequences with which it is immediately contiguous (in the 5′ and 3′ directions) in the naturally occurring genome of the organism from which it originates. For example, the “isolated nucleic acid” may comprise a DNA or cDNA molecule inserted into a vector, such as a plasmid or virus vector, or integrated into the DNA of a prokaryote or eukaryote.

With respect to RNA molecules of the invention, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (the term “substantially pure” is defined below).

The term “promoter region” refers to the transcriptional regulatory regions of a gene, which may be found at the 5′ or 3′ side of the coding region, or within the coding region, or within introns.

The term “vector” refers to a small carrier DNA molecule into which a DNA sequence can be inserted for introduction into a host cell where it will be replicated. An “expression vector” is a specialized vector that contains a gene with the necessary regulatory regions needed for expression in a host cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “substantially pure” refers to a preparation comprising at least 50-60% by weight the compound of interest (e.g., nucleic acid, oligonucleotide, protein, etc.). More preferably, the preparation comprises at least 75% by weight, and most preferably 90-99% by weight, the compound of interest. Purity is measured by methods appropriate for the compound of interest (e.g. chromatographic methods, agarose or polyacrylamide gel electrophoresis, HPLC analysis, and the like).

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the functional and novel characteristics of the sequence.

As used herein, the terms “reporter,” “reporter system”, “reporter gene,” or “reporter gene product” shall mean an operative genetic system in which a nucleic acid comprises a gene that encodes a product which when expressed produces a reporter signal that is readily measurable, e.g., by biological assay, immunoassay, radioimmunoassay, or by colorimetric, fluorogenic, chemiluminescent or other method. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, and may include, but not be limited to, such elements as promoters, enhancers, translational control sequences, poly A addition signals, transcriptional termination signals and the like.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like. The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on to or inherited by progeny cells or organisms of the recipient cell or organism. In other applications, the introduced nucleic acid may exist in the recipient cell or host organism only transiently.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and the method used. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is anti-parallel to the first region if the residue is thymine or uracil. Similarly, it is known that a cytosine residue of a first nucleic acid strand is capable of base pairing with a residue of a second nucleic acid strand which is anti-parallel to the first strand if the residue is guanine. A first region of a nucleic acid is complementary to a second region of the same or a different nucleic acid if, when the two regions are arranged in an anti-parallel fashion, at least one nucleotide residue of the first region is capable of base pairing with a residue of the second region. Preferably, the first region comprises a first portion and the second region comprises a second portion, whereby, when the first and second portions are arranged in an anti-parallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A first region of an oligonucleotide “flanks” a second region of the oligonucleotide if the two regions are adjacent one another or if the two regions are separated by no more than about 1000 nucleotide residues, and preferably no more than about 100 nucleotide residues.

A second set of primers is “nested” with respect to a first pair of primers if, after amplifying a nucleic acid using the first pair of primers, each of the second pair of primers anneals with the amplified nucleic acid, such that the amplified nucleic acid can be further amplified using the second pair of primers.

As used herein, an “instructional material” includes a publication, a recording, a diagram, or any other medium of expression which can be used to communicate the usefulness of the composition of the invention for performing a method of the invention or for associating the presence of a C-allele of the FGF-3 gene in a mammal with carcinogenic susceptibility. The instructional material of the kit of the invention can, for example, be affixed to a container which contains a kit of the invention or be shipped together with a container which contains the kit. Alternatively, the instructional material can be shipped separately from the container with the intention that the instructional material and the kit be used cooperatively by the recipient.

### II. Single Nucleotide Polymorphism (SNP) in the FGF-3 Gene

In accordance with the present application, a SNP in the FGF-3 gene has been identified which is predictive of an individual's risk for certain types of cancer, particularly esophageal cancer.

Accordingly, the present invention provides a nucleic acid molecule comprising the sequence of SEQ ID NO: 1 and the complement thereof.

Nucleic acid molecules of the present invention may be prepared by two general methods: (1) Synthesis from appropriate nucleotide triphosphates, or (2) Isolation from biological sources. Both methods utilize protocols well known in the art.

The availability of nucleotide sequence information, such as a full length nucleic acid sequence having SEQ ID NO: 1, enables preparation of isolated nucleic acid molecules of the invention by oligonucleotide synthesis. Synthetic oligonucleotides may be prepared by the phosphoramidite method employed in the Applied Biosystems 38A DNA Synthesizer or similar devices. The resultant construct may be purified according to methods known in the art, such as high performance liquid chromatography (HPLC). Long, double-stranded polynucleotides, such as a DNA molecule of the present invention, must be synthesized in stages, due to the size limitations inherent in current oligonucleotide synthetic methods. Thus, for example, a 1.4 kb double-stranded molecule may be synthesized as several smaller segments of appropriate complementarity. Complementary segments thus produced may be annealed such that each segment possesses appropriate cohesive termini for attachment of an adjacent segment. Adjacent segments may be ligated by annealing cohesive termini in the presence of DNA ligase to construct an entire 1.4 kb double-stranded molecule. A synthetic DNA molecule so constructed may then be cloned and amplified in an appropriate vector.

Nucleic acid sequences of the present invention may also be isolated from appropriate biological sources using methods known in the art.

Also contemplated with the scope of the present invention are vectors or plasmids containing the nucleic acid sequence of SEQ ID NO: 1, and host cells or animals containing such vectors or plasmids. Methods for constructing vectors or plasmids containing the nucleic acid sequence of SEQ ID NO: 1, and host cells or animals containing the same are within the ability of persons skilled in the art of molecular biology.

### III. Genotyping of FGF-3 Gene

According to one aspect of the present invention, a method for determining whether a human is homozygous for the C-allele, heterozygous for the C- and T-alleles, or homozygous for the T-allele of the FGF-3 gene is provided. Substantially any method of detecting an allele of the FGF-3 gene, such as hybridization, amplification, restriction enzyme digestion, and sequencing methods, can be used.

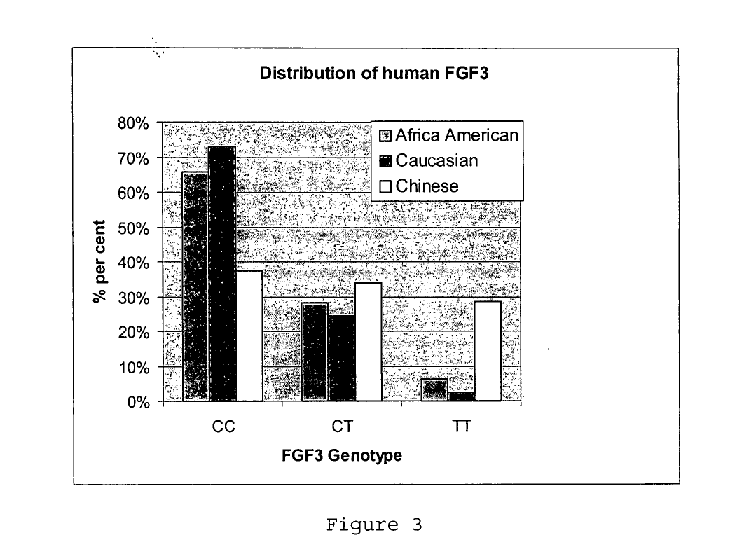

In one embodiment, an allelic discrimination method for identifying the FGF-3 genotype of a human was used (FIG. 3). The allelic discrimination method of the invention involves use of a first oligonucleotide probe which anneals with a target portion of the mammal's genome. The target portion comprises a portion of the upstream untranslated region of FGF-3 gene of the mammal, including the nucleotide residue at position 69 in SEQ ID NO: 1. Because the nucleotide residue at this position differs in the C-allele and the T-allele, the first probe is completely complementary to only one of the two alleles. Alternatively, a second oligonucleotide probe can also be used which is completely complementary to the target portion of the other of the two alleles. The allelic discrimination method of the invention also involves use of at least one, and preferably a pair of amplification primers for amplifying a reference region of the FGF-3 gene of the mammal. The reference region includes at least a portion of the target portion, and preferably includes the nucleotide residue at position 69 of the FGF-3 gene in SEQ ID NO: 1.

Because the reference region and the target portion overlap by at least one base, preferably by at least about half the length of the target portion, and more preferably completely overlap, the enzyme (e.g. Thermus aquaticus {Taq} DNA polymerase) which catalyzes the amplification reaction and the first (or second) probe will collide. If the probe is not completely complementary to the target portion, it is more likely to dissociate from the target portion upon collision than if it is completely complementary. Therefore, unless the enzyme exhibits 5′→3′ exonuclease activity, amplification ceases or is greatly inhibited.

If the enzyme which catalyzes the amplification reaction exhibits 5′→3′ exonuclease activity (e.g. Taq DNA polymerase), then the enzyme will at least partially degrade the 5′-end of a probe with which it collides unless the probe dissociates from the target portion upon collision with the enzyme. As noted above, if the probe is not completely complementary to the target portion, it is much more likely to dissociate from the target portion upon collision than if it is completely complementary. If a detectable label is attached to a nucleotide residue at or near the 5′-end of the probe, release of the detectable label from the probe can be used as an indication that the enzyme and probe have collided and that the probe did not dissociate from the target portion. Thus, release of the detectable label from the probe upon amplification of the region indicates that the probe was completely complementary to the target portion. By selecting either or both of a probe completely complementary to the target portion of the C-allele of the FGF-3 gene and a probe completely complementary to the target portion of the T-allele of the gene and assessing release of the label from the probe(s), the identity of the allele(s) can be ascertained.

The probe is preferably a DNA oligonucleotide having a length in the range from about 20 to about 40 nucleotide residues, preferably from about 20 to about 30 nucleotide residues, and more preferably having a length of about 25 nucleotide residues. In one embodiment, the probe is rendered incapable of extension by a PCR-catalyzing enzyme such as Taq polymerase, for example by having a fluorescent probe attached at one or both ends thereof. Although non-labeled oligonucleotide probes can be used in the kits and methods of the invention, the probes are preferably detectably labeled. Exemplary labels include radionuclides, light-absorbing chemical moieties (e.g. dyes), fluorescent moieties, and the like. Preferably, the label is a fluorescent moiety, such as 6-carboxyfluorescein (FAM), 6-carboxy-4,7,2′,7′-tetrachlorofluoroscein (TET), rhodamine, JOE (2,7-dimethoxy-4,5-dichloro-6-carboxyfluorescein), HEX (hexachloro-6-carboxyfluorescein), or VIC.

In a particularly preferred embodiment, the probe of the invention comprises both a fluorescent label and a fluorescence-quenching moiety such as 6-carboxy-N,N,N′,N′-tetramethylrhodamine (TAMRA), or 4-(4′-dimethlyaminophenylazo)benzoic acid (DABCYL). When the fluorescent label and the fluorescence-quenching moiety are attached to the same oligonucleotide and separated by no more than about 40 nucleotide residues, and preferably by no more than about 30 nucleotide residues, the fluorescent intensity of the fluorescent label is diminished. When one or both of the fluorescent label and the fluorescence-quenching moiety are separated from the oligonucleotide, the intensity of the fluorescent label is no longer diminished. Preferably, the probe of the invention has a fluorescent label attached at or near (i.e. within about 10 nucleotide residues of) one end of the probe and a fluorescence-quenching moiety attached at or near the other end. Degradation of the probe by a PCR-catalyzing enzyme releases at least one of the fluorescent label and the fluorescence-quenching moiety from the probe, thereby discontinuing fluorescence quenching and increasing the detectable intensity of the fluorescent labels. Thus, cleavage of the probe (which, as discussed above, is correlated with complete complementarity of the probe with the target portion) can be detected as an increase in fluorescence of the assay mixture.

If detectably different labels are used, more than one labeled probe can be used. For example, the assay mixture can contain a first probe which is completely complementary to the target portion of the C-allele of the FGF-3 gene and to which a first label is attached, and a second probe which is completely complementary to the target portion of the T-allele. When two probes are used, the probes are detectably different from each other, having, for example, detectably different size, absorbance, excitation, or emission spectra, radiative emission properties, or the like. For example, a first probe can be completely complementary to the target portion of the C-allele and have FAM and TAMRA attached at or near opposite ends thereof. The first probe can be used in the method of the invention together with a second probe which is completely complementary to the target portion of the T-allele and has TET and TAMRA attached at or near opposite ends thereof. Fluorescent enhancement of FAM (i.e. effected by cessation of fluorescence quenching upon degradation of the first probe by Taq polymerase) can be detected at one wavelength (e.g. 518 nanometers), and fluorescent enhancement of TET (i.e. effected by cessation of fluorescence quenching upon degradation of the second probe by Taq polymerase) can be detected at a different wavelength (e.g. 582 nanometers).

Ideally, the probe exhibits a melting temperature (Tm) within the range from about 60 to 70° C., and more preferably in the range from 65 to 67° C. Furthermore, because each probe is completely complementary to only one of the C- and T-alleles of the FGF-3 gene, each probe will necessarily have at least one nucleotide residue which is not complementary to the corresponding residue of the other allele. This non-complementary nucleotide residue of the probe is preferably located near the midsection of the probe (i.e. within about the central third of the probe sequence) and is preferably approximately equidistant from the ends of the probe. Thus, for example, the probe which is completely complementary to the C-allele of the human FGF-3 gene can, for example, be completely complementary to nucleotide residues 58 to 77 of the C-allele, as defined by the positions of SEQ ID NO: 1. Because the C- and T-alleles differ at position 69, this probe will have a mismatched base pair nine nucleotide residues from one end when it is annealed with the corresponding target portion of the T-allele.

By way of example, labeled probes having the sequences of SEQ ID NOs: 7 and 8 can be used, in conjunction with amplification primers having the sequences of SEQ ID NOs: 5 and 6 in order to determine the allelic content of a mammal (i.e. to assess whether the mammal comprises one or both of an C allele and a T allele of FGF-3).

The size of the reference portion which is amplified according to the allelic discrimination method of the invention is preferably not more than about 100 nucleotide residues. It is also preferred that the Tm for the amplified reference portion with the genomic DNA or fragment thereof be in the range from about 57 to 61° C., where possible.

It is understood that binding of the probe(s) and primers and that amplification of the reference portion of the FGF-3 gene according to the allelic discrimination method of the invention will be affected by, among other factors, the concentration of Mg⁺⁺ in the assay mixture, the annealing and extension temperatures, and the amplification cycle times. Optimization of these factors requires merely routine experimentation which are well known to skilled artisans.

Another allelic discrimination method suitable for use in the present invention employs “molecular beacons”. Detailed description of this methodology can be found in Kostrikis et al., Science 1998;279:1228-1229, which is incorporated herein by reference.

The use of microarrays comprising a multiplicity of reference sequences is becoming increasingly common in the art. Accordingly, another aspect of the invention comprises a microarray having at least one oligonucleotide probe, as described above, appended thereon. In a particular embodiment, the at least one oligonucleotide probe consists essentially of the nucleotide sequence of SEQ ID NO: 6 or SEQ ID NO: 7.

It is understood, however, that any method of ascertaining an allele of a gene can be used to assess the genotype of the FGF-3 gene in a mammal. Thus, the invention includes known methods (both those described herein and those not explicitly described herein) and allelic discrimination methods which may be hereafter developed.

### IV. Assessment of Predisposition to Cancer

The present invention also provides a method of assessing the relative susceptibility of a mammal (e.g. a human) to a cancer such as a esophageal squamous cell carcinoma. The “relative” susceptibility of a mammal to the cancer refers to the fact that, among a population of individuals exposed to equivalent carcinogenic stimuli, some individuals are more likely to develop cancers than others. This differential carcinogenic potential is attributable, at least in part to the genetic makeup of the individuals in the population. Germ-line mutations in tumor suppressor genes (e.g. Rb, p53, BRCA1, WT1, and the like) are one mechanism by which genetic differences contribute to predisposition to certain types of cancer. Mutations in other genes, such as oncogenes and genes encoding proteins involved in DNA repair, have also been implicated in carcinogenesis.

In accordance with the present invention, it has been discovered that the presence of a C-allele of the FGF-3 gene is correlated with greater susceptibility to carcinogenesis in mammals, particularly in humans. The effect is furthermore dose-dependent, meaning that a first individual who is homozygous for the C-allele has a greater susceptibility than a second individual who is heterozygous for the C- and T-alleles, and that the second individual has a greater susceptibility than a third individual who is homozygous for the T-allele. Thus, the method of the invention for assessing the relative susceptibility of a mammal to a cancer comprises determining whether the mammal comprises a C-allele, a T-allele or both in the 5′ untranslated region of the FGF-3 gene.

### V. Kit

The present invention also provides a kit for performing the instant method disclosed herein. The kit comprises a plurality of reagents useful for performing the disclosed methods, and optionally further comprises an instructional material which describes how the method is performed.

By way of example, an exemplary kit for performing the allelic discrimination method of the invention comprises:

a) a first oligonucleotide probe which anneals specifically with target portion of the mammal's genome, wherein the target portion includes the nucleotide residue located at position 69 of SEQ ID NO: 1, the probe comprising a fluorescent label and a fluorescence quencher attached to separate nucleotide residues thereof, and

b) a primer for amplifying a reference portion of the promoter region of FGF-3 gene, the reference portion including the nucleotide residue located at position 69 as defined by the sequence of SEQ ID NO: 1.

The kit may further comprise a DNA polymerase having 5′→3′ exonuclease activity. The kit may also comprise a second oligonucleotide probe having a different annealing specificity than the first (e.g. wherein the first is completely complementary to the target portion of the C-allele and the second is completely complementary to the target portion of the T-allele), a second primer (e.g. such that this and the other primer can be used to amplify at least the target portion by a PCR), or both. The kit may comprise an instructional material which can, for example, describe performance of the allelic discrimination method, the association between the presence of the C-allele and carcinogenic susceptibility, or both.

In an alternative embodiment of the present invention, the kit comprises at least one, and preferably two molecular beacon probes, as described herein. When the kit comprises two molecular beacon probes, one is preferably specific for (i.e. completely complementary to a region including nucleotide residue 69 of SEQ ID NO: 1) the C-allele of the FGF-3 gene, and the other is specific for the T-allele. This kit may further comprise an instructional material, as described above.

Also provided by the present invention are kits for assessing the susceptibility of a mammal to a cancer according to the one or more of the methods of the invention. The kit comprises a plurality of reagents useful for performing one of the methods as described above, and optionally further comprises an instructional material which describes how the method is performed and the association between the presence of the C-allele and carcinogenic susceptibility.

Although the foregoing disclosure is principally directed to kits and methods which are applicable to human cancers, it will be understood by the skilled artisan that such methods and kits are generally applicable to cancers of mammals of all sorts. Modification, where necessary, of the kits and methods of the invention to conform to non-human cancers is well understood, and the ordinarily skilled veterinary worker can design and perform such modification with merely ordinary, if any, experimentation. Representative mammals include, for example, primates, cattle, pigs, horses, sheep, cats, and dogs.

The kits and methods described herein are applicable for substantially any cancer, including, for example, esophageal carcinoma. Other cancers for which the compositions, kits, and methods described herein can be used include breast, ovarian, prostate, and head and neck cancer.

The following examples are provided to illustrate certain embodiments of the invention. They are not intended to limit the invention in any way.

### EXAMPLE I

### Identification of a Single Nucleotide Polymorphism (SNP) in the FGF-3 Gene

In this study, genomic DNA was obtained from 81 human subjects (39 Caucasians, 42 Chinese), and used for amplifying a 772 bp fragment which includes the 5′-proximal region of the FGF3 promoter. The primers used for this PCR procedure are as follows:

| | |
|---|---|
| Forward primer: | |
| 5′-gca, gcc, ctg, cct, cag, aaa, | (SEQ ID NO: 2) |
| ac-3′ | |
| Reverse primer: | |
| 5′-tgc, acc, cca, ctt, cta, gca, | (SEQ ID NO: 3) |
| tca, g-3′ | |

By direct sequencing of the 772 bp fragment, a single nucleotide polymorphism (SNP), C/T, was discovered at position 6693 bp upstream of the ATG start codon of FGF3 coding sequence, i.e., position 69 of SEQ ID NO: 1. (FIG. 1)

### EXAMPLE II

### Transfection Assays

To study the FGF-3 promoter activity for the C- and T-alleles of FGF-3 in vivo, two reporter constructs were generated. In these constructs, a fragment corresponding to nucleic acids 6761 to 6198 upstream of the ATG codon of human FGF-3 gene with either a C or T at position 6693 (SEQ ID NO: 1, FIG. 1) was cloned into pGL3 plasmid (Promega Corp., Madison, Wis) and operably linked with a modifier firefly luciferase gene. Using LIPOFECTIN Reagent (Life Technologies, Cat#: 18292-011), these C- or T-allele reporter constructs were transiently transfected into NIH3T3 cells. Luciferase activities were then assayed in cell extracts harvested 24 hours post-transfection. Cells transfected with empty pGL3 plasmid were used as controls. The results, listed in Table 2, reveal that the presence of the T-allele of the FGF-3 gene is associated with elevated promoter activity when compared with the C-allele.

TABLE 1
Functional analysis of the two human FGF-3
alleles

Luciferase activity^{a}

| Experiment | C allele | T allele |
|---|---|---|
| 1 | 31.03 | 92.49 |
| 2 | 14.90 | 23.64 |
| 3 | 46.38 | 73.20 |
| 4 | 18.93 | 26.73 |
| Mean ± SD | 27.8 ± 14.15 | 54.01 ± 34.23 |

^{a}Ratio of relative firefly luminescence units/μg of protein to relative renilla luminescence units/μg of protein measured in the same cell extract.

### EXAMPLE III

### Cancer Susceptibility Assessment

As described above, allelic discrimination methods, more specifically, TaqMan-based genotyping was used to determine the genotype of FGF-3 gene in human subjects. The following are the primers and probes used in these studies:

| | | |
|---|---|---|
| Primers: | | |
| Fgf3/snp #F: | 5′-gct, tca, ccc, cag, | (SEQ ID NO: 4) |
| | aga, tga, ggg-3′ | |
| Fgf3/snp #R: | 5′-agc, tgt, atg, cag, | (SEQ ID NO: 5) |
| | ccc, ctg, tg-3′ | |
| Probes: | | |
| Probe #1: | Vic-ctc, cct, cac, | (SEQ ID NO: 6) |
| | ctc, cag, cca, cat, | |
| | g-TAMRA | |
| Probe #2: | 6FAM-ctc, cct, cgc, | (SEQ ID NO: 7) |
| | ctc, cag, cca, cat, | |
| | g-TAMRA | |

By a TaqMan-based genotyping assay, the FGF-3 genotype distribution in DNA samples from 64 African American, 78 Caucasian, and 171 Chinese was determined. The results, shown in Table 2 and FIG. 3, clearly suggest that ethnic differences exist in FGF-3 allele frequencies.

TABLE 2
Distribution of Human FGF-3 Gene in Ethnic
Groups

| Group | CC | CT | TT | Total |
|---|---|---|---|---|
| Africa American | 42 | 18 | 4 | 64 |
| Caucasian | 57 | 19 | 2 | 78 |
| Chinese | 64 | 58 | 49 | 171 |
| Africa American | 66% | 28% | 6% | |
| Caucasian | 73% | 24% | 3% | |
| Chinese | 37% | 34% | 29% | |

A case-controlled study of esophageal cancer in China was also conducted by determining the distribution of C-allele FGF-3 gene among human subjects. In this study, the controls (n=171) were well matched on cases (n=168) for sex, age, and smoking status (Table 3). The results reveal an adjusted odds ratio (OR) of esophageal cancer of 2.38 for the CC genotype and 1.98 for the CT genotype.

TABLE 3
Characteristics of cases with esophageal
squamous cell carcinoma (ESCC) and controls

| Variable | Cases (n = 168) | Controls (n = 171) | P value |
|---|---|---|---|
| Sex (%) | | | 0.498 |
| Male | 144 (85) | 142 (83) | |
| Female | 24 (15) | 29 (17) | |
| Mean age (yr) (SD) | 58.1 (9.7) | 58.9 (5.0) | 0.480 |
| smoking status (%) | | | 0.645 |
| No | 61 (36) | 58 (34) | |
| Yes | 107 (46) | 113 (66) | |

TABLE 4
FGF-3 genotype in cases and controls and their
association with risk of ESCC

| | Cases | | Controls | | |
|---|---|---|---|---|---|
| | (n = 168) | | (n = 171) | | |
| Genotype | No. | % | No. | % | OR^{a,b,c} (95% CI^{d}) |
| TT | 26 | 15.5 | 49 | 28.7 | 1.00 |
| TC | 61 | 36.3 | 58 | 33.9 | 1.98 (1.15-3.83) |
| CC | 81 | 48.2 | 64 | 37.4 | 2.38 (1.32-4.29) |

| | | |
|---|---|---|
| C allele | 0.664 | 0.544 |
| frequency | | |

^{a}ORs and 95% CIs were calculated by logistic regression, with the TT genotype as the reference group and adjusted for age, sex, and smoking status.
^{b}Trend test, P = 0.005.
^{c}OR: odds ratio.
^{d}CI: confidence intervals.

The above results demonstrate that the presence of the C-allele in the FGF-3 gene 5′ untranslated region is correlated with greater susceptibility to esophageal cancer. Additionally, the effect is dose-dependent, i.e., a subject who is homozygous for the C-allele FGF-3 gene has a higher risk of developing esophageal cancer relative to subjects who are heterozygous (C-T). Finally, heterozygous individuals are at a greater risk than subjects who are homozygous for the T-allele at this position in the FGF-3 gene.

### REFERENCES

1. Dickson C et al, Nature 1987;326:833

2. Dickson C et al, Cell 1984;37:529-536

3. Nusse R, Trends Genet 1988;4:291-295

4. Galdemard C et al, Oncogene 1995;10(12):2331-2342

5. Suzuki H. et al, Cancer Invest 1997;15(5):411-5

6. Ikeda Y et al, Ann Thorac Surg. 1996;62(3):835-8

7. Yoshida T et al, Semin Cancer Biol. 1993;4(1):33-40

8. Kitagawa Y et al, Cancer Res. 1991;51(5):1504-8

9. Terada M et al, Princess Takamatsu Symp. 1991;22:371-80

10. Tsuda T et al, Cancer Res. 1989:49(20):5505-8

11. Naidu R et al, Int J Mol Med. 2001;8(2):193-8

12. Hajitou A et al, Breast Cancer Res Treat. 2000;60(1):15-28

13. Fioravanti L et al, Int J Cancer. 1997;74(6):620-4

14. An H X et al, Anticancer Res. 1997;17(4B):3133-6

15. Hui R et al, Oncogene. 1997;15(13):1617-23

16. Lonn U et al, Int J Cancer. 1996;69(4):273-7

17. Medl M et al, Gynecol Oncol. 1995;59(3):321-6

18. Esteller M et al, Cancer. 1995:75(8):2139-46

19. Champeme M H et al, Mod Pathol. 1994;7(9):900-5

20. Nagayama K et al, Nippon Geka Gakkai Zasshi. 1993;94(10):1144-52

21. Brookes S et al, Genes Chromosomes Cancer 1993;6(4):222-31

22. Ottestad L et al, Acta Oncol. 1993;32(3):289-94

23. Seki A et al, Int J Oncol. 2000;17(1):103-6

24. Schmitt J F et al, Growth Factors. 1996;13(1-2):19-35

25. Dobianer K et al, Gynecol Oncol. 1995;56(2):200-6

26. Rosen A et al, Br J Cancer. 1993;67(5):1122-5

27. Foulkes W D et al, Br J Cancer. 1993;67(2):268-73

28. Hruza C et al, Eur J Cancer. 1993;29A(11):1593-7

29. Sasano H et al, Hum Pathol. 1990;21(4):382-91

30. Ropiquet F et al, Cancer Res. 2000;60(15):4245-50

31. Fournier G et al, Urol Res. 1995;22(6):343-7

32. Latil A et al, Int J Cancer. 1994;59(5):637-8

33. Tseleni-Balafouta S et al, Eur J Endocrinol. 2002;146(l):57-60

34. Kleist B et al, J Oral Pathol Med. 2000;29(9):432-7

35. Fan C S et al, Hum Pathol. 2000;31(2):169-78

36. Schulte K M et al, Exp Clin Endocrinol Diabetes. 1996;104 Suppl 4:101-4

37. Lese C M et al, Genes Chromosomes Cancer. 1995;12(4):288-95

38. Rubin J S et al, J Laryngol Otol. 1995;109(1):72-6

39. Kitagawa Y et al, Cancer Res. 1991;51(5):1504-8

40. Djenabi, S. et al DNA Seq 1999;10(4-5):317-329,

41. Galdemard, C. et al, J Biol Chem 2000;275(23):17364-17373

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An isolated nucleic acid molecule comprising the sequence of SEQ ID NO: 1.

2. An isolated nucleic acid molecule comprising a sequence complementary to the sequence of claim 1.

3. A vector comprising the isolated nucleic acid molecule of claim 1, operably linked to a reporter gene.

4. The vector according to claim 3, wherein said reporter gene sequence encodes luciferase.

5. A host cell comprising the vector of claim 3.

6. A method for detection of a single nucleotide polymorphism (SNP) in the FGF-3 gene in a mammal, which method comprises:
a) isolating a nucleic acid sample from said mammal; and
b) determining whether a cytosine or thymine is present at position 69 of SEQ ID NO: 1.

7. The method according to claim 6, wherein the mammal is a human.

8. The method according to claim 6, wherein the determination of the presence of a cytosine or thymine comprises amplifying a reference portion of the mammal's genome.

9. The method according to claim 8, wherein the reference portion is amplified using a pair of primers consisting essentially of nucleotide sequences of SEQ ID NO: 4 and SEQ ID NO: 5.

10. The method according to claim 8, wherein the reference portion comprises the 5′ untranslated region of FGF-3 gene.

11. The method according to claim 10, wherein the 5′ untranslated region of FGF-3 gene comprises the nucleotide residue located at position 69 of SEQ ID NO: 1.

12. The method according to 8, further comprising annealing a first oligonucleotide probe with a target portion of the mammal's genome prior to amplifying the reference portion, wherein the target portion includes the nucleotide residue located at position 69 of SEQ ID NO: 1.

13. The method according to claim 12, wherein the first probe comprises a flourescent label.

14. The method according to claim 13, wherein the fluorescent label is selected from FAM, TET, rhodamine, VIC, JOE, and HEX.

15. The method according to claim 13, wherein the first probe further comprises a fluorescence quencher.

16. The method according to claim 15, wherein the quencher is selected from TAMRA and DABCYL.

17. The method according to claim 12, wherein the first probe consists essentially of the nucleotide sequence of SEQ ID NO: 6.

18. The method according to claim 15, wherein the reference portion is amplified using a DNA polymerase having 5′→3′ exonuclease activity.

19. The method according to claim 12, further comprising annealing a second oligonucleotide probe with said target portion of the mammal's genome prior to amplifying the reference portion, wherein said first probe is completely complimentary to the target portion of T-allele FGF-3 gene and said second probe is completely complimentary to the target portion of C-allele FGF-3 gene.

20. The method according to claim 19, wherein said second probe consists essentially of the nucleotide sequence of SEQ ID NO: 7.

21. The method according to claim 19, wherein said first probe comprises a first fluorescence label and said second probe comprises a second fluorescence label, said first and second fluorescence labels being detectably different.

22. The method according to claim 21, wherein said first and second fluorescence labels are selected from the group consisting of FAM, TET, rhodamine, VIC, JOE, and HEX.

23. The method according to claim 21, wherein said first and second probes further comprises a first and second fluorescence quencher, respectively.

24. The method according to claim 23, wherein said first and second fluorescence quenchers are selected from the group consisting of TAMRA and DABCYL.

25. A kit for performing the method according to claim 6 comprising:
a) a first oligonucleotide probe which anneals specifically with a target portion of the mammal's genome, wherein said first probe comprises a first fluorescent label and a first fluorescence quencher attached to separate nucleotide residues thereof and said target portion includes the nucleotide residue located at position 69 of SEQ ID NO: 1; and
b) a pair of primers for amplifying a reference portion of the FGF-3 gene, wherein said reference portion includes the nucleotide residue located at position 69 of SEQ ID NO: 1.

26. The kit according to claim 25 further comprising a DNA polymerase having 5′→3′ exonuclease activity.

27. The kit according to claim 26, further comprising a second oligonucleotide probe, wherein said first probe is completely complementary to said target portion if the nucleotide residue located at position 69 of SEQ ID NO: 1 is cytosine, and said second oligonucleotide probe is completely complementary to said target portion if the nucleotide residue located at position 69 of SEQ ID NO: 1 is thymine.

28. The kit according to claim 27 further comprising an instructional material.

29. A method of assessing the relative susceptibility of a mammal to cancer, said method comprising the detection of the SNP in FGF-3 gene according to claim 6, wherein if the mammal comprises nucleotide cytosine at position 69 of SEQ ID NO: 1, then the mammal has a greater susceptibility to the cancer than a mammal of the same type which does not comprise nucleotide cytosine at position 69 of SEQ ID NO: 1.

30. The method according to claim 29, wherein said the mammal is a human.

31. The method according to claim 30, wherein the cancer is selected from the group consisting of esophageal, breast, ovarian, prostate, and head and neck cancer.

32. The method according to claim 31, wherein the esophageal cancer is esophageal squamous cell carcinoma.

33. A microarray having at least one oligonucleotide probe that can anneal with a target portion of a mammal's genome, wherein the target portion includes the nucleotide residue located at position 69 of SEQ ID NO: 1.

34. The microarray according to claim 33, wherein said at least one oligonucleotide probe consists essentially of nucleotide sequences selected from the group consisting of SEQ ID NOs: 6 and 7.


SEQUENCE LISTING (7 sequences)

SEQ ID NO: 1 - DNA, 564 bp, Homo sapien
  misc_feature (0)...(0): n is cytosine or thymine
gcagccctgcctcagaaaacagaaggacgcagcacactcacggtgactcacccccatgtggctggaggngagggagcctcctgaggcagggccagggcagccgtcaggtgggtgacggcaggggtcttgccatggtgggcacaggggctgcatacagcttactcagtgacaatcgagtccctggtgccagcctctggaagtctggaagtgagcaatgtttcccattaaggaaagtgtgtggccggccatgccccccaacgttgcacactcactgcctttgcagggttggggcttccagtcacagggtcccatccacgtaccagcccaggtggctgcagaaggtccctcgcagtcatgaaaccaagggaggcttgggaaaccacatctgaagggcatggctttgatttagtgagagggtggggctgggctgggcaaggccaccaggtctgagtcagagccagaggcaggaagctggtccccagcactgcccgccgcctctgcgatgcagtcctcctggccacctgagaacagcctgtagagaggcagtggcgtctttcggacttc

SEQ ID NO: 2 - DNA, 20 bp, Artificial Sequence
  primer
gcagccctgcctcagaaaac

SEQ ID NO: 3 - DNA, 22 bp, Artificial Sequence
  primer
tgcaccccacttctagcatcag

SEQ ID NO: 4 - DNA, 21 bp, Artificial Sequence
  primer
gcttcaccccagagatgaggg

SEQ ID NO: 5 - DNA, 20 bp, Artificial Sequence
  primer
agctgtatgcagcccctgtg

SEQ ID NO: 6 - DNA, 22 bp, Artificial Sequence
  probe
ctccctcacctccagccacatg

SEQ ID NO: 7 - DNA, 22 bp, Artificial Sequence
  probe
ctccctcgcctccagccacatg